(19) United States
(12) Patent Application Publication
(10) Pub. No.: US 2012/0089023 A1
(43) Pub. Date: Apr. 12, 2012

(54) PARAMETER-SENSING ENDOTRACHEAL TUBE

(76) Inventors: DAN SCHLAGER, TIBURON, CA (US); DELMAS R. BUCKLEY, JR., LIVERMORE, CA (US)

(21) Appl. No.: 13/158,395

(22) Filed: Jun. 11, 2011

Related U.S. Application Data
(63) Continuation-in-part of application No. 12/901,491, filed on Oct. 9, 2010.

(51) Int. Cl. A61B 8/12; A61B 5/087; A61B 5/08

(52) U.S. Cl. 600/439; 600/529; 600/538; 600/532

(57) ABSTRACT

A sensor-equipped endotracheal tube has a flexible body surrounding an airway lumen and includes sensors for monitoring physiological parameters such as CO₂/O₂ concentration in respiratory gases, and patient body temperature. These sensors provide electrical output for parameter display on suitable ventilators and monitors. In another specific embodiment, the endotracheal tube includes a sensor for measuring pressure exerted between an inflation cuff and a patient's tracheal tissues for preventing injury from over/under cuff inflation. Other specific embodiments of the sensor equipped endotracheal tube include combining sensors with a heating member for warming inhaled respiratory gases, and with ultrasound reflecting elements that make the tube visible within the body using ultrasound.

### RELATED APPLICATION AND CLAIM OF PRIORITY

This non-provisional U.S. patent application is a continuation-in-part of co-pending U.S. patent application Ser. No. 12/901,491 filed 2010 Oct. 9 entitled ULTRASOUND-OBSERVABLE, RESPIRATORY GAS-WARMING, PARAMETER-SENSING ENDOTRACHEAL TUBE. This patent application claims priority from the filing date of said co-pending patent application pursuant to the provisions of 35 U.S.C. §120.

New matter introduced in this continuation-in-part is found at the BRIEF DESCRIPTION OF THE DRAWING section and includes new drawing FIGS. 9a, 9b and 10. Additional new matter continues at the end of the original DESCRIPTION OF THE PREFERRED EMBODIMENT section following the description of original FIG. 8c.

### FIELD OF THE INVENTION

The invention relates generally to endotracheal tubes, and more particularly, to tubes including elements, singly and in combination, for improving ultrasound observability, for the warming of inhaled gases, and for the measurement of physiological parameters such as inflation cuff pressure and CO₂/O₂ concentration levels of respiratory gases.

### BACKGROUND ART

Endotracheal intubation is the placement of a flexible plastic tube through the open mouth and into the trachea (windpipe) in order to maintain an open airway in patients who are unconscious or unable to breathe on their own. Oxygen, anesthetics, or other gaseous medications can be delivered through the tube using a self-filling bag and valve (bag valve) type hand operated pump/bellows or an automated mechanical ventilator.

Prior Art FIG. 1 illustrates a patient 10 intubated with an endotracheal tube 12 such as that illustrated in U.S. Pat. No. 6,378,523. An inserted end (the distal end) 14 of the endotracheal tube is positioned within the patient's trachea 16, and includes an inflatable cuff 18 used to seal the airway after the tube is properly positioned.

The endotracheal tube 12 is typically placed into a patient with the aid of a laryngoscope (not illustrated), a hand-held device that permits the health care professional to view the larynx while aligning the endotracheal tube during insertion. The method is not without its difficulties, and there is a risk of serious injury to the throat, and in some cases of prolonged interruption of breathing, injury to the brain, and even death resulting from lack of oxygen. These risks are particularly pronounced in emergency situations.

U.S. Pat. No. 7,543,586 (the '586 patent) suggests the usefulness of ultrasound for observing the endotracheal tube within the patient. Unlike the laryngoscope which is inserted through the open mouth of a patient and allows a view only so far as the larynx, ultrasound permits views of the inner structures of the neck from outside the body, in theory providing the health care professional information useful to insure the tube is properly and safely inserted.

The '586 patent illustrates use of a wire stylete (‘stilette’ in the '586 patent) inserted through the tube's airway to both stiffen and guide the plastic tube during insertion. An intubating stylet is a malleable metal wire, among a class of devices that are inserted into the airway of an endotracheal tube to make the tube conform better to the anatomy of the specific individual, thus facilitating a safe insertion. The wire stylete of the '586 patent ends at a small metal ball located at the inserted end of the wire. The metal ball is primarily intended to prevent injury to the patient caused by the inserted end of the wire stylete, and in the '586 patent, serves also as an ultrasound target.

The intubating stylete has drawbacks, as do all the devices of this class, one of which is that its presence in the tube's airway prevents the attachment of a hand operated bag valve or of a mechanical ventilator during the intubation procedure. Only after the tube has been properly positioned in the patient and the wire stylete withdrawn can ventilation begin providing oxygen to the patient. As a result, use of a stylete risks a delayed airflow to the lungs during the critical intubation process, causing some health care professionals to avoid use of the intubating stylete and the other guidance devices of a similar nature. Another drawback is that the metal used to form a typical wire stylete is not sufficiently ultrasound reflective to make the wire easily observable within the patient; a point not made in the '586 patent.

The flexible plastic body of a typical endotracheal tube, of which FIG. 1 is a representative example, is also not easily observable within the patient using ultrasound for assistance during intubation. What is needed is an ultrasound reflective plastic body that makes the tube readily observable by the health care professional during a tracheal intubation to insure the tube enters the trachea (windpipe), and not the esophagus (carries food and water to the stomach), an error all too common with existing methods.

At times it is desirable to warm the gases being applied to an intubated patient. The typical endotracheal tube is basically a plastic tube incapable of warming the inhalation gases. It would be useful to have an endotracheal tube with a heating member for warming inhalation gases.

For endotracheal tubes equipped with an inflatable cuff near the distal end for sealing the trachea once intubation is achieved, there is always a danger that over inflation of the cuff may cause injury to the patient, while under inflation can result in regurgitated stomach content being forced into the lungs. Such accidents are especially prone to occur in emergency situations and during patient transport, such as in an ambulance or helicopter. It would be useful if the cuff-equipped endotracheal tube included a sensor for measuring the pressure between the inflated cuff and the tissues of the patient's trachea. This pressure measurement would help alleviate these problems.

Finally, no known endotracheal tube includes a sensor for measuring CO₂ concentration of exhaled gases. At times it is critical to know such concentration while a patient is intubated. It would be desirable to have an endotracheal tube including a sensor for measuring CO₂ concentration of exhaled breath of an intubated patient.

### SUMMARY OF THE INVENTION

These needs, and others that will become apparent, are met by specific embodiments of an endotracheal tube that include at least one of the following elements: an ultrasound-observable flexible body, a heating member for warming inhalation gases, a sensor for measuring pressure between an inflation cuff and patient trachea tissues, and sensors for measuring CO₂ and O₂ concentration levels in respiratory gases; wherein all of these elements, alone and in various useful combinations, do not impede a normal flow of respiratory gases through the endotracheal tube.

### BRIEF DESCRIPTION OF THE DRAWING

FIG. 1 is a partial pictorial diagram that illustrates a patient intubated with a common prior art endotracheal tube before attachment of a ventilating device.

FIG. 2 is a partial pictorial diagram illustrating a portion of the patient of FIG. 1, and the use of an ultrasound observable endotracheal tube according to a specific embodiment of the present invention.

FIG. 3 is a partial pictorial diagram illustrating a portion of an endotracheal tube that is ultrasound reflective.

FIG. 4 is a partial pictorial diagram illustrating a portion of an endotracheal tube having ultrasound reflective bands surrounding the body according to a specific embodiment of the invention.

FIG. 5a through 5g are partial pictorial diagrams that show various embodiments of ultrasound observable enhancements according to specific embodiments of the invention.

FIG. 6a through 6d are partial pictorial diagrams illustrating endotracheal tubes including internal heating members for warming inhalation gases.

FIG. 7 is a partial pictorial diagram that illustrates an endotracheal tube having a proximal heating member for warming inhalation gases.

FIG. 8a through 8c are partial pictorial diagrams illustrating an endotracheal tube including a sensor for measuring a physiological parameter such as cuff pressure or CO₂/O₂ concentration.

FIGS. 9a, 9b are partial pictorial diagrams illustrating an endotracheal tube having a concentric construction of flexible plastic tubes, and at least one flexible tube being ultrasound reflecting.

FIG. 10 is a partial pictorial diagram illustrating a sensor equipped endotracheal tube connected to a medical ventilator or, alternatively, to a monitor.

### DESCRIPTION OF THE PREFERRED EMBODIMENT

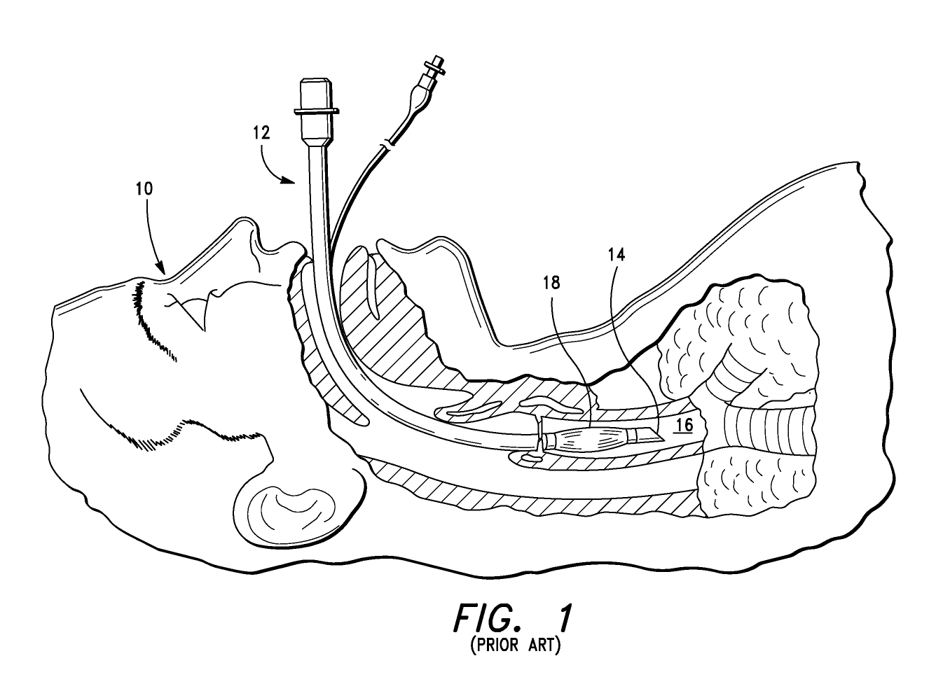

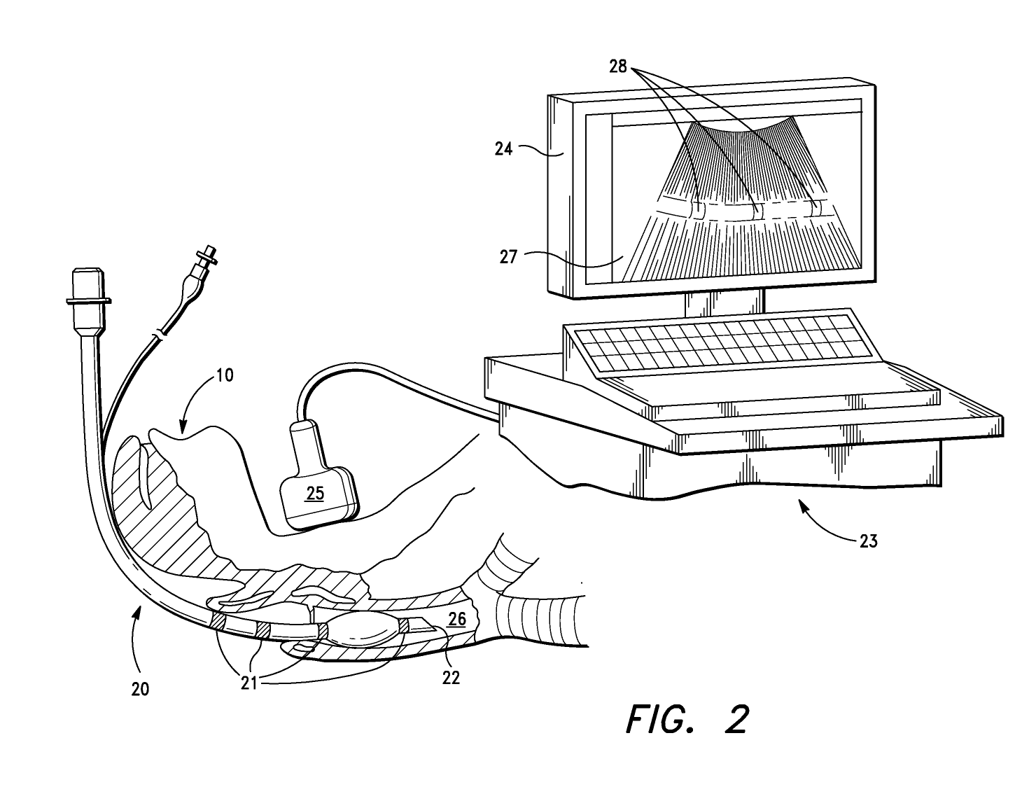

With reference to FIG. 2 there is shown a partial pictorial diagram illustrating a portion of the patient 10 of the prior art FIG. 1, and the use of an ultrasound observable endotracheal tube 20 according to a specific embodiment of the present invention. The patient 10 is intubated with the endotracheal tube 20 having ultrasound-reflecting elements 21 disposed near a distal end 22. Also shown is an ultrasound machine 23 including a display 24 and a hand-held ultrasound wand 25. The endotracheal tube 20 has been properly inserted into the patient's trachea 26 with the aid of ultrasound for observing the location of the tube within the patient's throat. An ultrasound image 27 includes a portion of the endotracheal tube 20 including stripes 28, corresponding to the ultrasound-reflecting elements 21.

The hand held ultrasound wand 25 is located approximately above the patient's laryngeal prominence (Adam's apple). Experience teaches that this region is a likely place to view the ultrasound-reflecting elements 21 while the endotracheal tube is being inserted. Viewing by placing the hand-held wand 25 at the side of the patient's neck is also recommended because of the trachea is located in front of the esophagus within the neck, as more clearly seen in prior art FIG. 1 where the trachea is above the esophagus of the prone patient.

In general, the plastic tube itself is not visible using ultrasound. Rather, some ultrasound-reflecting element must be placed within or upon the tube to permit it to be seen in the ultrasound image 27 during intubation. What appears in the ultrasound image 27 to be a tube is actually an image of body tissues displaced by the tube and conforming along the tube's edges. The tube itself is difficult to see directly because a typical plastic material used to make an endotracheal tube of the type illustrated is not a good reflector of ultrasound. Experience has also taught that tissue conformance alone is not generally helpful while guiding the endotracheal tube to a proper and safe location within the trachea.

A solution to the problem presented by various embodiments of the present invention is to make the flexible plastic endotracheal tube ultrasound reflective. Note that in the description that follows, the phrases ‘ultrasound-reflecting’ and ‘ultrasound reflective’ are used interchangeably and are not intended to define different features or characteristics.

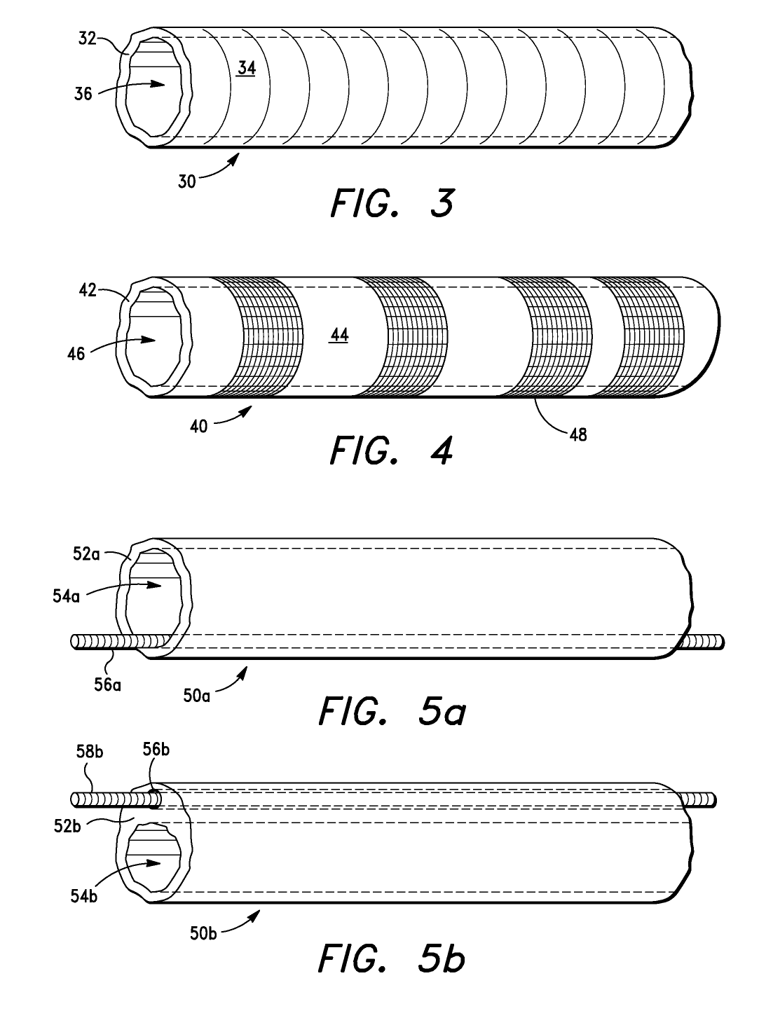

In a specific embodiment of the invention, the plastic material of the tube itself is made ultrasound reflective, as illustrated in FIG. 3, a partial pictorial diagram of a portion of an endotracheal tube, indicated generally by the reference numeral 30. The tube 30 includes a flexible plastic body 32, having an outer surface 34, and an airway passage, indicated generally by the reference numeral 36, extending from one end of the tube to the other. The plastic material out of which the tube is constructed includes an ultrasound-reflecting element (not illustrated because the body material itself is ultrasound reflective).

In an alternative embodiment, the material of the tube is not ultrasound reflective; instead, an ultrasound reflective tape is applied to an outer surface of the tube. This specific embodiment is illustrated in FIG. 4, a partial pictorial diagram of a portion of an endotracheal tube, indicated generally by the reference numeral 40. The tube 40 includes a flexible plastic body 42, having an outer surface 44, and an airway passage, indicated generally by the reference numeral 46, extending from one end of the tube to the other. The flexible plastic body 42 has a limited response or a non-reflective response to ultrasound, and is made ultrasound reflective by the addition of strips 48 of ultrasound reflective tape disposed upon the outer surface 44.

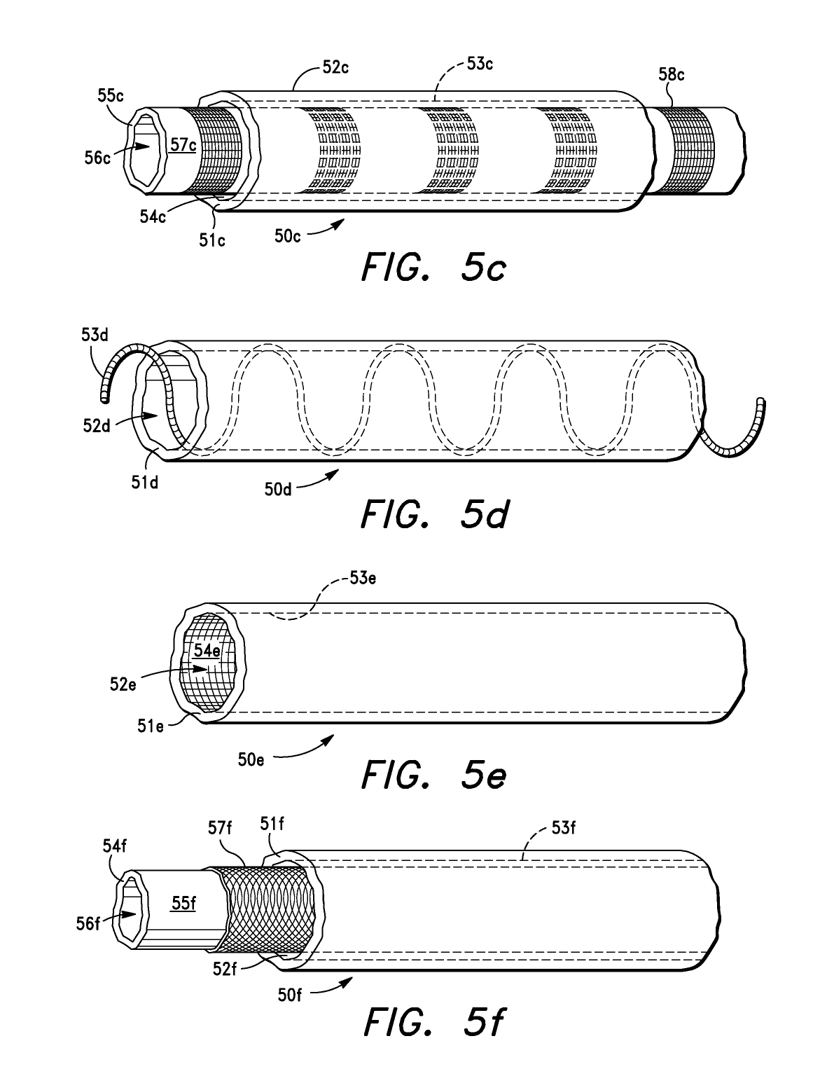

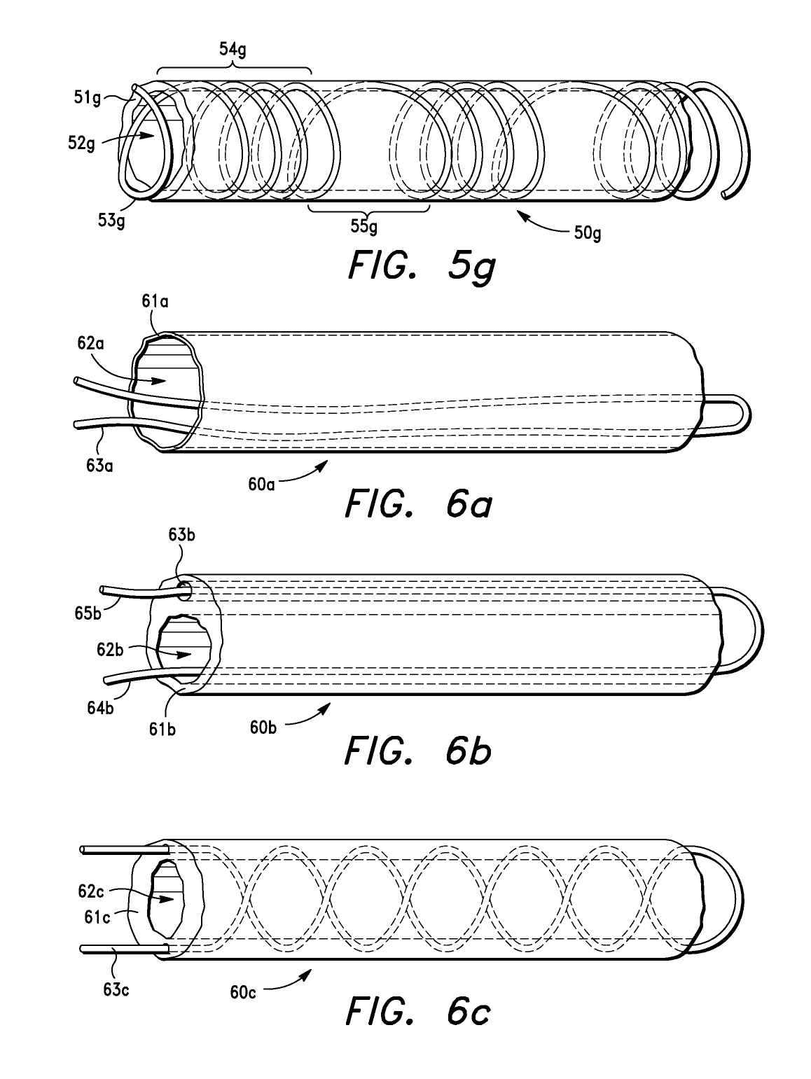

FIGS. 5a through 5g are partial pictorial diagrams that illustrate a typical portion of an endotracheal tube according to specific embodiments of the invention having an ultrasound reflective member located inside the body of the tube in various ways. FIG. 5a illustrates a portion of an endotracheal tube according to a specific embodiment of the present invention. The tube is indicated generally by the reference numeral 50a, including a flexible plastic body 52a, having an airway passage indicated generally by the reference numeral 54a. Extending within the airway passage 54a is a metallic structure 56a which can serve a variety of purposes: as an ultrasound-reflecting element, and as a stiffening element for assisting the flexible body 52a to be formable into a shape making insertion of the tube less likely to cause harm to a patient. The metal of structure 56a is selected to enhance ultrasound reflectivity.

FIG. 5b illustrates a portion of an endotracheal tube according to another specific embodiment of the present invention. The tube is indicated generally by the reference numeral 50b, including a flexible plastic body 52b, having an airway passage indicated generally by the reference numeral 54b, and a secondary lumen 56b through which passes a metallic structure 58b.

FIG. 5c illustrates a portion of an endotracheal tube of concentric construction according to yet another specific embodiment of the present invention. The tube is indicated generally by the reference numeral 50c, including a first flexible plastic body 51c, having an outer surface 52c, an inner surface 53c, a first lumen 54c, a second flexible plastic body 55c fitted snugly within the first lumen, the second flexible body having a second lumen, indicated generally by the reference numeral 56c and forming a airway passage, an outer surface 57c, and ultrasound-reflecting elements 58c forming bands disposed upon the outer surface 57c. A person having an ordinary level of skill in the art will appreciate that a description of the endotracheal tube 50c stating that an inner tube fits snugly within an outer tube will result in the same structure as a description stating that an outer tube fits snugly over an inner tube. In another specific embodiment, the ultrasound-reflecting member(s) 58c are disposed between an inner surface of an outer tube and an outer surface of an inner tube. In yet another specific embodiment, the ultrasound-reflecting element(s) 58c comprise a reflective tape.

FIG. 5d illustrates a portion of an endotracheal tube including an ultrasound-reflecting element according to another embodiment of the invention. The endotracheal tube is indicated generally by the reference numeral 50d and includes a flexible body 51d having an airway-lumen indicated generally by the reference numeral 52d and a metallic construction 53d molded into the flexible body and providing an ultrasound-reflecting element. In another specific embodiment, the metallic construction 53d is a wire coiled about the airway-lumen and molded into the flexible body, as illustrated.

FIG. 5e illustrates a portion of an endotracheal tube having an ultrasound-reflecting element disposed upon an inner surface of an airway-lumen, and indicated generally by the reference numeral 50e. The tube 50e includes a flexible body 51e having an airway-lumen indicated generally by the reference numeral 52e, the airway-lumen has an inner surface 53e, and the tube has an ultrasound-reflecting element 54e disposed upon the inner surface.

FIG. 5f illustrates a portion of an endotracheal tube of concentric construction and indicated generally by the reference numeral 50f. The tube 50f includes a first flexible body 51f having a first lumen, indicated generally by the reference numeral 52f, the first lumen having an inner surface 53f. The endotracheal tube 50f also includes a second flexible body 54f having an outer surface 55f and an airway-lumen, indicated generally by the reference numeral 56f. The second flexible body fits snugly within the lumen of the first flexible body, and an ultrasound-reflecting element 57f is disposed between the two flexible bodies. The ultrasound-reflecting element is a metallic construction and in a specific embodiment comprises a braided wire shield.

FIG. 5g illustrates a portion of an endotracheal tube having a construction similar to that illustrated in FIG. 5d, and is indicated generally by the reference numeral 50g. The tube includes a flexible body 51g having an airway-lumen, indicated generally by the reference numeral 52g. The tube also includes an ultrasound-reflecting element 53g which in a specific embodiment is a metallic construction molded into the flexible body. The metallic construction is coiled within the flexible body and about the airway-lumen, as illustrated, and spacing of the metallic coils is adapted for making possible via use of ultrasound an estimate of tube insertion distance within a patient. In FIG. 5g the coils are grouped into tightly spaced coil regions 54g that are separated by loosely spaced regions 55g.

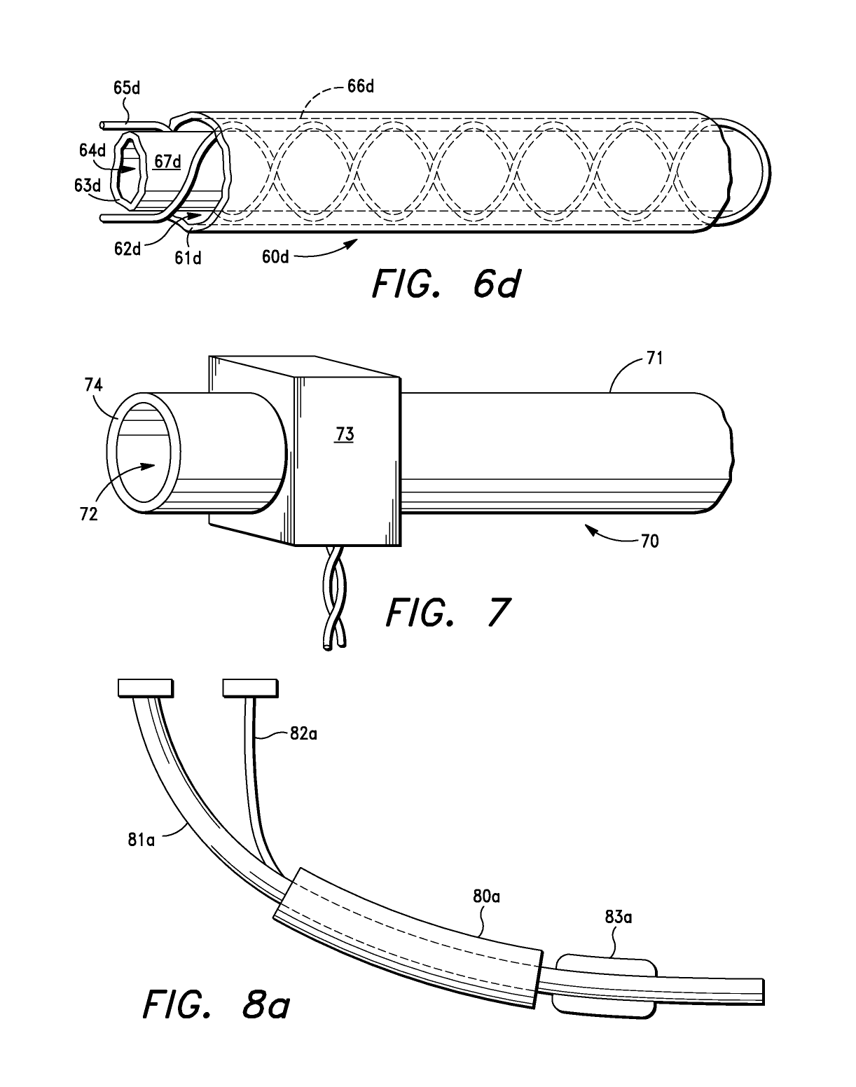

FIGS. 6a through 6d are partial pictorial diagrams that illustrate a typical portion of an endotracheal tube according to specific embodiments of the invention in which a heating member located within the body of the tube heats inhaled respiratory gases moving through the tube.

FIG. 6a illustrates a portion of an endotracheal tube, indicated generally by the reference numeral 60a, having a flexible body 61a, an airway-lumen, indicated generally by the reference numeral 62a, and a heating member 63a disposed within the airway-lumen. The heating member warms a respiratory gas moving through the airway-lumen. In FIG. 6a the heating member is a metallic wire and warming is produced by passing an electric current through the wire.

In FIG. 6b the endotracheal tube is indicated generally by the reference numeral 60b and includes a flexible body 61b having an airway-lumen, indicated generally by the reference numeral 62b, and a secondary lumen 63b. In a specific embodiment of the invention, a heating member 64b is disposed in the airway-lumen and returns 65b in the secondary lumen, as illustrated.

FIG. 6c illustrates a typical portion of an endotracheal tube having a heating member molded into a flexible body. The tube is indicated generally by the reference numeral 60c and includes a flexible body 61c having an airway-lumen, indicated generally by the reference numeral 62c. A heating member 63c is coiled within the flexible body about the airway-lumen, as illustrated. Passing an electric current through the heating member warms respiratory gases moving through the airway-lumen. Though the coils appear evenly spaced in FIG. 6c, a person having an ordinary level of skill in the art will appreciate that the coil spacing can be adapted to allow estimation of insertion distance of the tube into a patient, as in FIG. 5g.

FIG. 6d illustrates a typical portion of an endotracheal tube having a heating member disposed in coils within a concentric construction. The tube is indicated generally by the reference numeral 60d, and includes a first flexible body 61d having a first lumen, indicated generally by the reference numeral 62d, a second flexible body 63d having an airway-lumen, indicated generally by the reference numeral 64d. A heating member 65d is coiled between an inner surface 66d of the first lumen and an outer surface 67d of the second flexible body. In a specific embodiment, the first flexible body, the heating member, and the second flexible body fit snugly together to make possible a safe intubation.

FIG. 7 is a partial pictorial diagram showing a portion of an endotracheal tube including a heating member located at the proximal end (outside the patient). The tube is indicated generally by the reference numeral 70. The tube includes a flexible body 71 having an airway-lumen, generally indicated by the reference numeral 72. A heating member 73 is located near a proximal end 74 of the tube, and thus remains external to the patient when the tube is intubated. A valve (not illustrated) located within the heating member is operated by the direction of a respiratory gas. When the gas is moving into the patient (inhalation), the gas passes through the heating member and is warmed. When the gas is moving in the opposite direction, out of the patient (exhalation), the valve operates to bypass the heating member. This arrangement permits a normal hand operated bag valve or a mechanical ventilator to remain attached at the proximal end of the tube (not illustrated) while the heating member warms inhaled respiratory gases.

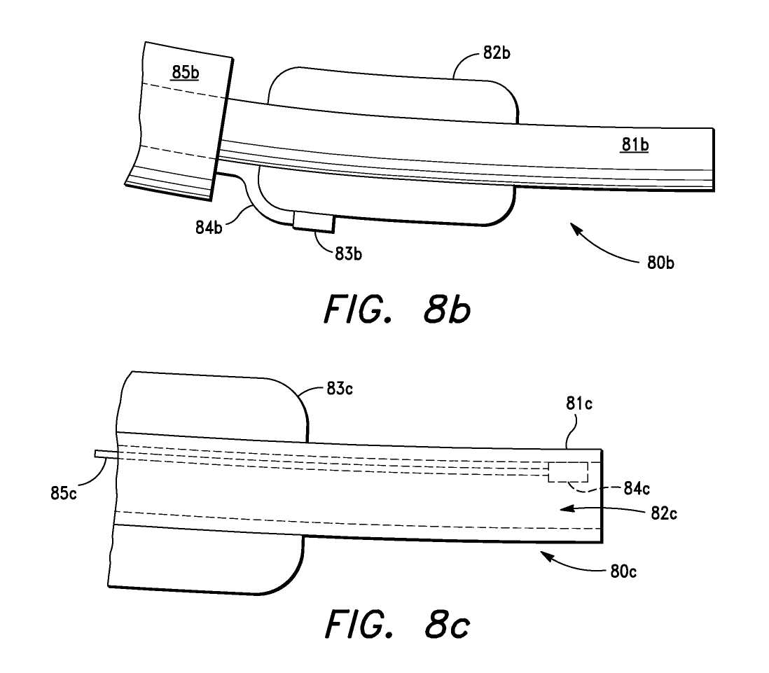

FIGS. 8a through 8c are partial pictorial diagrams illustrating a portion of an endotracheal tube having an inflatable cuff for sealing the trachea once insertion is achieved and a sensor for measuring a physiological parameter such as temperature, pressure, or gaseous concentration. Though the appearance of the inflatable cuff in FIGS. 8a, 8b and 8c differs from the appearance of the cuff illustrated in FIG. 2, the difference has no significance.

FIG. 8a illustrates such a tube having a protective sheath 80a surrounding a main breathing tube 81a and secondary tubes and/or wires/fiber optics 82a, and an inflatable cuff 83a. A purpose of the sheath is to protect the secondary tubes, wires, fiber optics and a patient from becoming entangled. The diameter of the protective sheath is exaggerated with respect to the diameter of the endotracheal main breathing tube, and in reality fits snugly about the breathing tube to facilitate successful intubation.

FIG. 8b illustrates a pressure sensor disposed upon an outer surface of the inflatable cuff for measuring pressure between an inflated cuff and patient's trachea. A problem that arises often, particularly in emergency situations, with use of an inflatable cuff is that it can easily become overinflated, causing injury to a patient. The endotracheal tube is indicated generally by the reference numeral 80b, and includes a main breathing tube 81b, an inflatable cuff 82b, a pressure sensor 83b, connecting wires 84b, and protective sheath 85b. As the inflatable cuff is inflated, pressure between the cuff and a patient's tracheal tissues (see FIG. 2) increases. Sometimes during emergency intubations a patient's tracheal tissues become injured as a result of inadvertent over-inflation of the cuff. In a specific embodiment, the pressure sensor 83b measures a pressure between the cuff and a patient's surrounding tissue and reports the measured pressure value via the electrical wires 84b.

FIG. 8c illustrates a sensor located on an inner surface of the airway-lumen near the distal end of the tube. Such placement is useful for measuring temperature of a respiratory gas and gaseous concentration levels. The endotracheal tube is indicated generally by the reference numeral 80c and includes a flexible body 81c having an airway-lumen indicated generally by the reference numeral 82c, an inflatable cuff 83c, a sensor 84c and wires 85c for transmitting a sensor measured value outside the endotracheal tube and patient. A variety of sensors are useful for this embodiment including a thick film conductive type CO₂ sensor, a nanotube molecular wires chemical sensor, and a fiber optic chemical sensor such as that disclosed in U.S. Pat. No. 4,842,783, the full disclosure of which is incorporated herein by reference. When a fiber optic is used to implement the CO₂ sensor, it will be appreciated that the element 85c, previously referred to as a wire, is instead a fiber optic for carrying the sensor measurement back to a user. In an alternative embodiment of the invention, the sensor 84c is used for sensing O₂ concentration levels in respiratory gases passing through the airway-lumen 82c.

### NEW MATTER

In other embodiments (not illustrated), a sensor located near the distal end of the tube is used to measure patient body temperature. In one such embodiment, the temperature sensor is located on an outer surface of the inflatable cuff (e.g., sensor 83b of FIG. 8b). In another such embodiment, the temperature sensor is located on an outer surface of the tube 81b (FIG. 8b). In yet another such embodiment, the temperature sensor is located on an inner surface of the airway-lumen (e.g., sensor 84c of FIG. 8c). Electrical wires from the sensor connect back to a display device, such as part of a ventilator or a monitor. In other embodiments (not illustrated), multiple sensors are located near the distal end of the tube for measuring cuff pressure, body temperature, and CO₂/O₂ concentration. These sensors are connected via electrical wires (and in some embodiments by optical fiber) back to display devices, such as part of a ventilator.

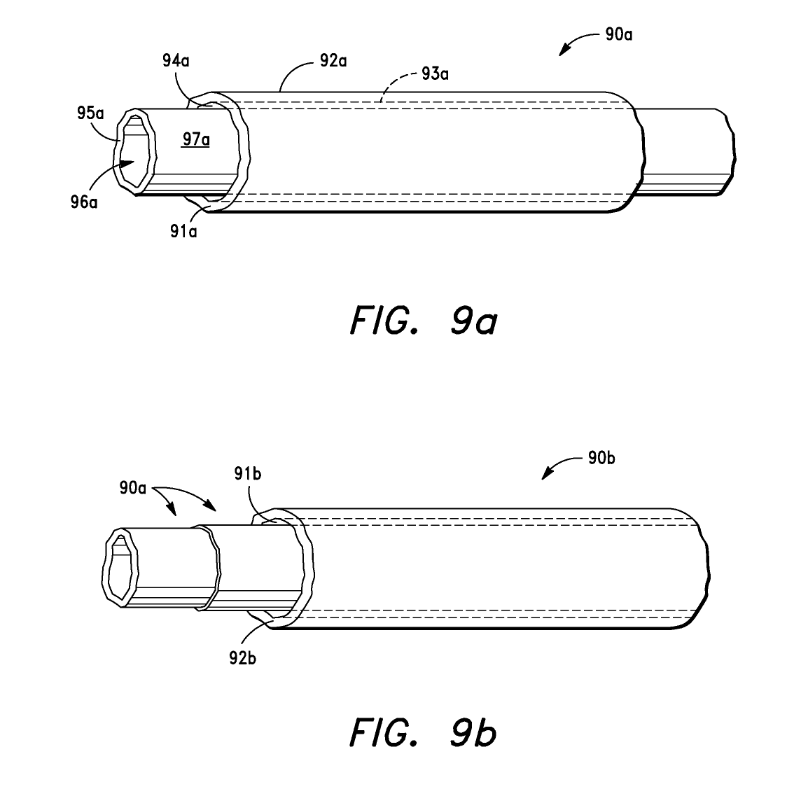

FIGS. 9a and 9b are partial pictorial diagrams that illustrate an endotracheal tube made of a concentric construction and in which at least one flexible tube of the construction is made of an ultrasound reflective material.

FIG. 9a illustrates a portion of an endotracheal tube of concentric construction according to yet another embodiment of the present invention. The tube is indicated generally by the reference numeral 90a, and includes a first flexible plastic body 91a, having an outer surface 92a, an inner surface 93a, a first lumen 94a, a second flexible plastic body 95a fitted snugly within the first lumen, the second flexible body having a second lumen, indicated generally by the reference numeral 96a and forming a airway passage, and an outer surface 97a. At least one of the first and second flexible bodies is made of an ultrasound reflective plastic. In another embodiment, at least one of the flexible bodies is made of an ultrasound opaque material. A person having an ordinary level of skill in the art will appreciate that a description of the endotracheal tube 90a stating that an inner tube fits snugly within an outer tube will result in the same structure as a description stating that an outer tube fits snugly over an inner tube.

FIG. 9b illustrates a portion of an endotracheal tube of concentric construction according to yet another embodiment of the present invention. The tube is indicated generally by the reference numeral 90b, and includes the two-layer structure illustrated in FIG. 9a, indicated generally by reference numeral 90a, fitted snugly within a lumen 91b of an outer flexible plastic body 92b. At least one flexible body of the tube 90b is made of an ultrasound reflective plastic. In another embodiment, at least one of the flexible bodies is made of an ultrasound opaque material.

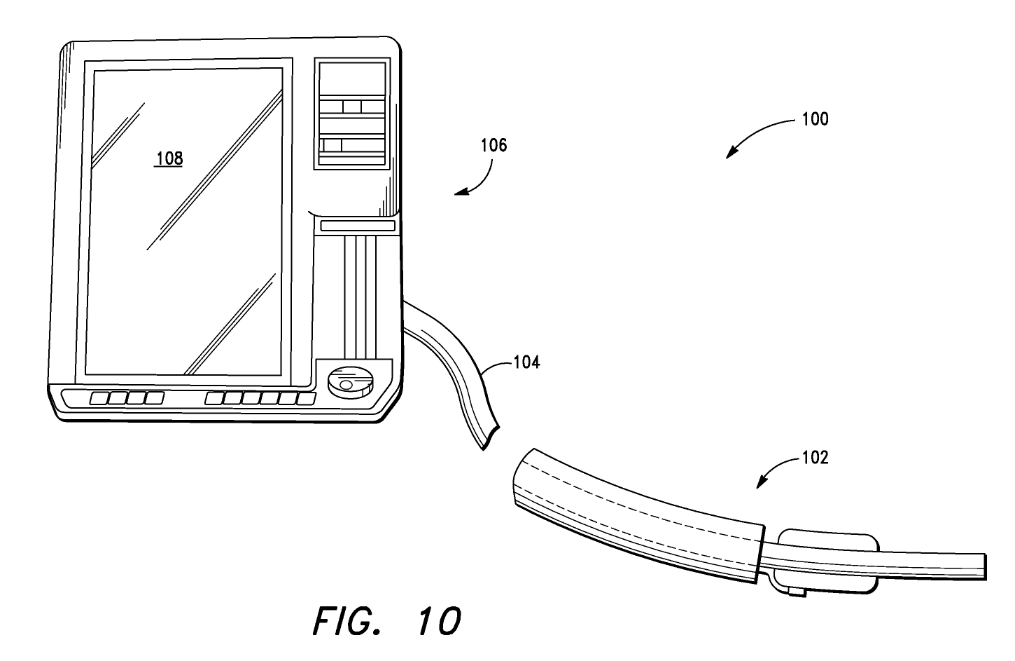

FIG. 10 illustrates a ventilating and parameter sensing/display system 100 including a parameter-sensing endotracheal tube 102 connected via air-way tube and electrical wires (and in some embodiments by optical fiber) 104 to a ventilator 106 having a display 108 for displaying sensed parameters including inflation cuff pressure, body temperature, and CO₂/O₂ concentration. The system is compatible for use with the parameter-sensing endotracheal tubes illustrated in FIGS. 3-10. In some embodiments in which the endotracheal tube will be used in a high-energy field, for example, on an intubated patient within an MRI or CT-scan machine, the use of copper wires for connecting sensors back to a display is replaced by titanium wires (not illustrated). In some scenarios, the parameter-sensing endotracheal tube 102 is electrically connected to a monitor 106 for display of parameters. In other scenarios, the patient is ventilated using a self-filling bag and valve (bag valve) type hand operated pump/bellows (not illustrated).

While the invention has been described in relation to the embodiments shown in the accompanying Drawing figures, other embodiments, alternatives and modifications will be apparent to those skilled in the art. It is intended that the Specification be exemplary only, and that the true scope and spirit of the invention be indicated by the following Claims.

1. A sensor equipped endotracheal tube, comprising:
a. a flexible body having an outer surface, proximal and distal ends, and adapted for patient intubation;
b. an airway-lumen, open at both ends, extending through the flexible body between the proximal and distal ends; and
c. a sensor for measuring a parameter.

2. The endotracheal tube of claim 1, wherein the sensor is adapted for permitting a free passage of respiratory gas through the airway-lumen.

3. The endotracheal tube of claim 1, further including an inflatable cuff located near the distal end of the flexible tube, a secondary tube for inflating and deflating the cuff, wherein the sensor measures a pressure between the cuff and patient trachea tissue.

4. The endotracheal tube of claim 3, further including an ultrasound-reflecting element.

5. The endotracheal tube of claim 3, further including a respiratory gas-heating member.

6. The endotracheal tube of claim 3, further including an ultrasound-reflecting element and a respiratory gas-heating member.

7. The endrotracheal tube of claim 1, wherein the sensor measures patient body temperature.

8. The endotracheal tube of claim 7, further including an ultrasound-reflecting element.

9. The endotracheal tube of claim 7, further including a respiratory gas-heating member.

10. The endotracheal tube of claim 7, further including an ultrasound-reflecting element and a respiratory gas-heating member.

11. The endotracheal tube of claim 1, further including electrical wires for making an electrical connection with the sensor.

12. The endotracheal tube of claim 11, wherein the electrical wires are made of titanium.

13. The endotracheal tube of claim 1, wherein the sensor measures a parameter related to a respiratory gas flowing through the airway-lumen.

14. The endotracheal tube of claim 13, wherein the sensor measures a CO₂ concentration of an exhaled respiratory gas.

15. The endotracheal tube of claim 14, further including an ultrasound-reflecting element.

16. The endotracheal tube of claim 14, further including a respiratory gas-heating member.

17. The endotracheal tube of claim 14, further including an ultrasound-reflecting element and a respiratory gas-heating member.

18. The endotracheal tube of claim 14, further including an inflatable cuff located near the distal end of the flexible tube, a secondary tube for inflating and deflating the cuff, and a pressure sensor for measuring a pressure between the cuff and patient trachea tissue.

19. The endotracheal tube of claim 18, further including an ultrasound-reflecting element.

20. The endotracheal tube of claim 18, further including a respiratory gas-heating member.

21. The endotracheal tube of claim 18, further including an ultrasound-reflecting element and a respiratory gas-heating member.

22. The endotracheal tube of claim 13, wherein the sensor measures an O₂ concentration of a respiratory gas.

23. The endotracheal tube of claim 22, further including an ultrasound-reflecting element.

24. The endotracheal tube of claim 22, further including a respiratory gas-heating member.

25. The endotracheal tube of claim 22, further including an ultrasound-reflecting element and a respiratory gas-heating member.

26. The endotracheal tube of claim 22, further including an inflatable cuff located near the distal end of the flexible tube, a secondary tube for inflating and deflating the cuff, and a pressure sensor for measuring a pressure between the cuff and patient trachea tissue.

27. The endotracheal tube of claim 26, further including an ultrasound-reflecting element.

28. The endotracheal tube of claim 26, further including a respiratory gas-heating member.

29. The endotracheal tube of claim 26, further including an ultrasound-reflecting element and a respiratory gas-heating member.

30. An ultrasound observable endotracheal tube having a concentric construction, comprising:
a. a first flexible body having an outer surface, an inner surface, proximal and distal ends, and adapted for patient intubation;
b. a first lumen, open at both ends, extending through the first flexible body between the ends;
c. a second flexible body fitting snugly within the first lumen and extending from the proximal to distal ends of the first flexible body;
d. a second lumen, open at both ends, extending through the second flexible body between its ends, defining an airway; and
e. an ultrasound-reflecting member disposed between distal and proximal ends of the flexible bodies.

31. The endotracheal tube of claim 30, wherein the ultrasound-reflecting member is a metallic construction further disposed between the inner surface of the first flexible body and the outer surface of the second flexible body.

32. The endotracheal tube of claim 30, wherein the ultrasound-reflecting member is an ultrasound reflective tape further disposed between the inner surface of the first flexible body and the outer surface of the second flexible body.

33. The endotracheal tube of claim 30, wherein at least one flexible body is made of an ultrasound reflective material, said at least one flexible body defining the ultrasound-reflecting member.

34. The endotracheal tube of claim 33, wherein the ultrasound reflective material is an ultrasound reflective plastic.

35. The endotracheal tube of claim 33, wherein the ultrasound reflective material is a plastic including an ultrasound reflective compound.

36. The endotracheal tube of claim 30, wherein the ultrasound-reflecting member is a third flexible body having a lumen extending between proximal and distal ends, the third flexible body being disposed between the inner surface of the first flexible body and the outer surface of the second flexible body.

37. The endotracheal tube of claim 30, further including a sensor for measuring a parameter.

38. The endotracheal tube of claim 37, wherein the sensor measures one of a body temperature, a CO₂ concentration of a respiratory gas, and an O₂ concentration of a respiratory gas.

39. The endotracheal tube of claim 37, further including an inflatable cuff located near the distal end of the flexible tube, a secondary tube for inflating and deflating the cuff, and wherein the sensor measures a pressure between the cuff and patient trachea tissue.

40. The endotracheal tube of claim 39, further including a respiratory gas-heating member.
